(19) United States
(12) Patent Application Publication
(10) Pub. No.: US 2019/0049384 A1
(43) Pub. Date: Feb. 14, 2019

(54) HIGH-SENSITIVITY LATERAL FLOW IMMUNOASSAY STRIP BASED ON SURFACE-ENHANCED RAMAN SCATTERING AND DETECTION METHOD USING THE SAME

(71) Applicant: INDUSTRY-UNIVERSITY COOPERATION FOUNDATION HANYANG UNIVERSITY ERICA CAMPUS, Ansan-si, Gyeonggi-do (KR)

(72) Inventors: Jaebum CHOO, Ansan-si, Gyeonggi-do (KR); Sangyeop LEE, Ansan-si, Gyeonggi-do (KR); Joonki HWANG, Gangneung-si, Gangwon-do (KR)

(73) Assignee: INDUSTRY-UNIVERSITY COOPERATION FOUNDATION HANYANG UNIVERSITY ERICA CAMPUS, Ansan-si, Gyeonggi-do (KR)

(21) Appl. No.: 15/760,328

(22) PCT Filed: Sep. 23, 2016
(86) PCT No.: PCT/KR2016/010688

§ 371 Date: Mar. 15, 2018

(30) Foreign Application Priority Data
Sep. 23, 2015 (KR) 10-2015-0134753
Sep. 22, 2016 (KR) 10-2016-0121330

(51) Int. Cl. G01N 21/65; G01N 21/78; G01N 33/558; G01N 33/543

(52) U.S. Cl. CPC G01N 21/658; G01N 33/54346; G01N 33/558; G01N 21/78

(57) ABSTRACT

The present disclosure relates to a surface-enhanced Raman scattering (SERS) lateral flow immunoassay strip containing: a sample pad into which a sample containing a target material is introduced; a conjugate pad containing a hollow metal nanoprobe for surface-enhanced Raman scattering, on which an antibody that can be coupled to the target material and a Raman marker are immobilized; and a detection pad including a detection region to which a secondary antibody that can be coupled to the target material coupled to the hollow metal nanoprobe is immobilized. Use of the SERS-based lateral flow immunoassay strip according to the present disclosure enables high-sensitivity quantitative analysis and qualitative analysis of the target material from Raman signal measurement depending on the concentration of the target material.

### CROSS-REFERENCE TO RELATED APPLICATION

This application claims priority to and the benefit of Korean Patent Applications Nos. 2015-0134753 and 2016-0121330, filed on Sep. 23, 2015 and Sep. 22, 2016, respectively, the disclosures of which are incorporated herein by reference in its entirety.

### BACKGROUND

### 1. Field of the Invention

The present disclosure relates to a lateral flow immunoassay strip capable of qualitative analysis and high-sensitivity quantitative analysis of a target material based on surface-enhanced Raman scattering (hereinafter referred to as ‘SERS’) and a method for detecting a target material using the same.

### 2. Description of Related Art

Lateral flow immunoassay (LFA) is an analytical technique capable of detecting a target material in an unknown sample based on the sandwich immunoassay technique using nanoparticles and flow of the sample using a membrane.

FIG. 1 shows a diagnostic strip commonly used in lateral flow immunoassay. As seen from FIG. 1, a general diagnostic strip contains a rectangular support (not shown) made of an adhesive plastic material and a sample pad, a conjugate pad, a detection pad and an absorption pad disposed sequentially on the support from one end to the other end.

The lateral flow immunoassay is used for diagnosis or nonmedical self-test in various medical/environmental fields because it is simple in principle, enables the analysis of a target material in short time and can be produced at low cost.

The lateral flow immunoassay is widely used in on-site analysis because it is inexpensive, is portable, enables fast detection and can be easily used by unskilled laypeople. A typical example using the lateral flow immunoassay is a pregnancy test kit for detecting human chorionic gonadotropin (hCG) from urine.

In the lateral flow immunoassay, the hCG introduced to the sample pad is conjugated to a gold nanoparticle-antibody conjugate immobilized on the conjugate pad and flows along the membrane (detection pad) by a capillary phenomenon. As a result of conjugation to a secondary antibody immobilized on a detection region, the gold nanoparticle, which is a detection marker (probe), develops color.

In the lateral flow immunoassay, detection is made visually based on the color development by the gold nanoparticle which forms the immune complex with the target material. For precise diagnosis and detection of the target material, a reading system capable of high-sensitivity analysis and quantitative analysis through improvement in analytical technique is necessary.

However, the lateral flow immunoassay does not have good analysis sensitivity because it is based on visual assessment and quantitative analysis is difficult. In addition, it is difficult to detect samples requiring higher sensitivity because the lateral flow immunoassay has low sensitivity. Furthermore, the analytical technique cannot be applied to samples requiring quantitative analysis.

Surface-enhanced Raman scattering (SERS)-based detection is an analytical method that can overcome the detection sensitivity limit of Raman spectroscopy. This analytical method can quantify a target material by measuring the change in the intensity of characteristic SERS peaks amplified by a Raman reporter molecule (Raman marker). When the reporter molecule is adsorbed on a rough metal surface and is exposed to an excitation light (laser light), SERS signals are increased remarkably due to electromagnetic and chemical resonance at the SERS active site of the reporter molecule known as a “hot junction” (non-patent document 1). This resonance effect is expected to solve the low sensitivity problem of the existing Raman method and to overcome the accuracy and detection limits of the existing chemiluminescence assay and radiation-based immunoassay.

The inventors of the present disclosure have completed the present disclosure by developing an SERS-based high-sensitivity lateral flow immunoassay technique through consistent researches.
(Non-patent document 1) Kneipp, J. et al., 1997. Phys. Rev. Lett. 78, pp. 1667-1670.

### SUMMARY OF THE INVENTION

The present disclosure is directed to providing a surface-enhanced Raman scattering (SERS)-based lateral flow immunoassay strip.

The present disclosure is also directed to providing a method for detecting a target material using a SERS-based lateral flow immunoassay strip.

The present disclosure is also directed to providing a SERS-based lateral flow immunoassay kit.

The present disclosure relates to a SERS-based lateral flow immunoassay strip sensor. The basic principle of the SERS-based lateral flow immunoassay strip sensor of the present disclosure is the same as that of the existing POC (point of care)-based lateral flow immunoassay strip. The difference of the present disclosure from the existing immunoassay strip is as follows. The existing POC-based lateral flow immunoassay strip uses a general metal nanoprobe and is not based on SERS measurement. In contrast, the present disclosure uses a Raman marker-coupled hollow metal nanoprobe and SERS measurement is performed.

That is to say, the present disclosure applies a hollow metal nanoprobe for SERS measurement to an immunoassay strip sensor. The application of the hollow metal nanoprobe for SERS measurement to the immunoassay strip sensor enables the qualitative detection of the presence of a target material through color change of a detection region in a detection pad and the quantitative analysis of the target material by measuring the intensity of SERS signals.

In the present disclosure, the “SERS-based lateral flow immunoassay strip sensor” is also called a “SERS-based lateral flow immunoassay strip”, a “SERS-based LFA strip sensor”, a “SERS-based LFA strip” or a “SERS-based LFA”.

Also, in the present disclosure, the “POC-based lateral flow immunoassay strip sensor” also called a “POC-based LFA strip sensor”, a “POC-based LFA strip” or a “POC-based LFA” refers to a LFA strip sensor based on visual detection without using SERS measurement, in comparison with the “SERS-based LFA strip” of the present disclosure.

The present disclosure provides a SERS-based lateral flow immunoassay strip, containing:

a sample pad into which a sample containing a target material is introduced;

a conjugate pad containing a hollow metal nanoprobe for surface-enhanced Raman scattering, on which an antibody that can be coupled to the target material and a Raman marker are immobilized;

and a detection pad including a detection region to which a secondary antibody that can be coupled to the target material coupled to the hollow metal nanoprobe is immobilized.

The target material may be detected by identifying color development at the detection pad and measuring a SERS signal.

The SERS-based lateral flow immunoassay strip may further contain an absorption pad present in a general immunoassay strip.

The target material refers to a material to be detected and includes a protein (antigen), a nucleic acid, a small molecule, etc.

The detection pad may further include a control region to which an antibody that is coupled to the hollow metal nanoprobe is immobilized. The control region is located downstream of the detection region along the flow direction of the sample. The antibody adsorbed on the control region is an antibody that can be coupled directly to the antibody on the metal nanoprobe for SERS measurement regardless of the presence of a target material (antigen). For example, it may be IgG.

The detection region is also called a test line T and the control region is also called a control line C.

The detection of the target material may include qualitative analysis of identifying the presence of the target material through color development of the detection region and quantitative analysis of identifying the amount of the target material by measuring a SERS signal.

The detection limit of the target material may be 0.001 ng/mL or lower.

The hollow metal nanoprobe used in the present disclosure is described in detail in Korean Patent Registration No. 10-0979727.

The hollow metal nanoprobe may be a hollow gold nanoparticle or a hollow gold nanosphere (HGN). Methods for preparing the hollow gold nanoparticle are described in detail in Korean Patent Registration No. 10-0979727 and Korean Patent Publication No. 10-2012-0017358. These literatures and disclosures of the literatures are incorporated herein by reference.

In the present disclosure, the “Raman marker” coupled to the metal nanoprobe refers to a Raman reporter molecule and any one known in the art may be used. Examples may include X-rhodamine-5-isothiocyanate (XRITC), crystal violet (CV) or malachite green isothiocyanate (MGITC), although not being limited thereto.

In another aspect, the present disclosure provides a method for detecting a target material using the SERS-based lateral flow immunoassay strip.

Specifically, the present disclosure provides a method for detecting a target material using a SERS-based lateral flow immunoassay strip, including:

a step of introducing a sample containing a target material into a sample pad;

and a step of identifying color development at the detection pad and measuring a SERS signal.

The detection of the target material may include qualitative analysis of identifying the presence of the target material through color development of a detection region of the detection pad and quantitative analysis of identifying the amount of the target material by measuring a SERS signal.

FIG. 2 (a) schematically describes the qualitative identification of the presence of a target material by the existing lateral flow immunoassay. However, the existing lateral flow immunoassay strip sensor has the problem that quantitative analysis is impossible. The SERS-based immunoassay strip according to the present disclosure solves this problem and enables quantitative analysis through SERS signal measurement while allowing qualitative analysis as the existing method.

A detailed description is given below. The sample dropped on the sample pad moves toward the conjugate pad, where the target material is conjugated to an antibody on the hollow metal nanoprobe to form a primary immune complex of target material-hollow metal nanoparticle. Then, the target material-hollow metal nanoprobe immune complex moves toward the detection pad and is conjugated to a secondary antibody of the detection region to form a secondary (sandwich) immune complex of secondary antibody-target material-hollow metal nanoprobe.

The amount of the secondary immune complex formed in the detection region (test line) increases with the amount of the target material and the test line develops a red line due to the plasmonic signal of the accumulated nanoparticle. The hollow metal nanoprobe on which the unreacted antibody is immobilized moves further and is coupled to an antibody adsorbed on a control region (control line). As a result, both the test line and the control line turn red and develop red lines in the presence of the target material, whereas only the control line develops a red line in the absence of the target material. Therefore, qualitative analysis is possible.

FIG. 2 (b) describes an analysis platform of the SERS-based immunoassay strip sensor of the present disclosure. The nanoparticle used in the SERS-based immunoassay strip of the present disclosure is a hollow metal nanoparticle on which a Raman marker is adsorbed. It is used for quantitative analysis of an antigen to be detected. In the presence of an antigen, the nanoparticle is accumulated in the test line and the test line develops a red line. Quantitative analysis of the concentration of the antigen is possible using a SERS signal from the Raman marker on the surface of the accumulated nanoparticle.

In another aspect, the present disclosure provides a SERS-based lateral flow immunoassay kit containing: the SERS-based lateral flow immunoassay strip; and a SERS signal detector.

The SERS signal detector may be any one widely known in the art.

In an exemplary embodiment of the present disclosure, high-sensitivity detection is achieved by lateral flow immunoassay using the hollow metal nanoprobe for amplifying the Raman signal. In addition, the signal is acquired with high reproducibility by applying the surface-enhanced Raman scattering mapping technique.

Use of a SERS-based lateral flow immunoassay strip according to the present disclosure enables, in addition to qualitative analysis of identifying the presence of a target material, high-sensitivity quantitative analysis of the concentration of the target material from Raman signal measurement. Specifically, a kit according to the present disclosure provides 100-1,000 times improved sensitivity as compared to the existing technique.

The present disclosure realizes high-sensitivity quantitative analysis using the intensity of an optical signal depending on the amount of the target material, in addition to the advantage of fast detection based on visual detection. The SERS-based lateral flow immunoassay strip according to the present disclosure can be used for testing in clinical setting, environmental analysis, screening for food sanitation, etc. because it enables fast detection, high reproducibility through SERS mapping and high-sensitivity quantitative analysis.

### BRIEF DESCRIPTION OF THE DRAWINGS

FIG. 1 shows a diagnostic strip commonly used in lateral flow immunoassay.

FIG. 2 compares the existing lateral flow immunoassay (a) with SERS-based high-sensitivity lateral flow immunoassay according to the present disclosure (b).

FIG. 3 shows the TEM images (a), UV/VIS absorption spectrum (b) and DLS distribution (c) of a hollow gold nanoparticle (HGN).

FIG. 4 shows a DLS signal from an antibody physically (a) or chemically (b) immobilized on HGN.

FIG. 5 shows the SEM image of a HGN immune complex when 10 ng/mL SEB is present on a test line (a) and the SEM image of a HGN immune complex when SEB is absent (b).

FIG. 6 shows the quantitative analysis result of a target material through SERS mapping using a lateral flow immunoassay strip according to an exemplary embodiment of the present disclosure.

FIG. 7 shows the result of visual detection and detection by enzyme-linked immunosorbent assay (ELISA) of SEB at different concentrations.

FIG. 8 compares the sensitivity of SERS-based lateral flow immunoassay according to an exemplary embodiment of the present disclosure and the existing technique.

FIG. 9 shows the images of a SERS-based lateral flow immunoassay strip of the present disclosure and the result of SERS mapping when SEB, SEA (Staphylococcus aureus enterotoxin A), ochratoxin, aflatoxin and fumonisin are present.

FIG. 10 shows the SERS mapping images of SEB at low concentrations (500, 100, 50, 10 and 1 ng/mL) (a), LFA images (b) and quantitative analysis result obtained from curve fitting of the SERS mapping images (c). The effect of nonspecific conjugation was tested using an antigen cocktail containing five different antigens (SEB, SEA, ochratoxin, aflatoxin and fumonisin).

FIG. 11 compares the Raman intensity of a test line of a SERS-based LFA strip using HGN (hollow gold nanoparticle) (a) and a SERS-based LFA strip using GNP (gold nanoparticle) depending on the concentration of SEB.

FIG. 12 shows the calibration curves for quantitative analysis of a SERS-based LFA strip using HGN (a) and a SERS-based LFA strip using GNP depending on the concentration of SEB.

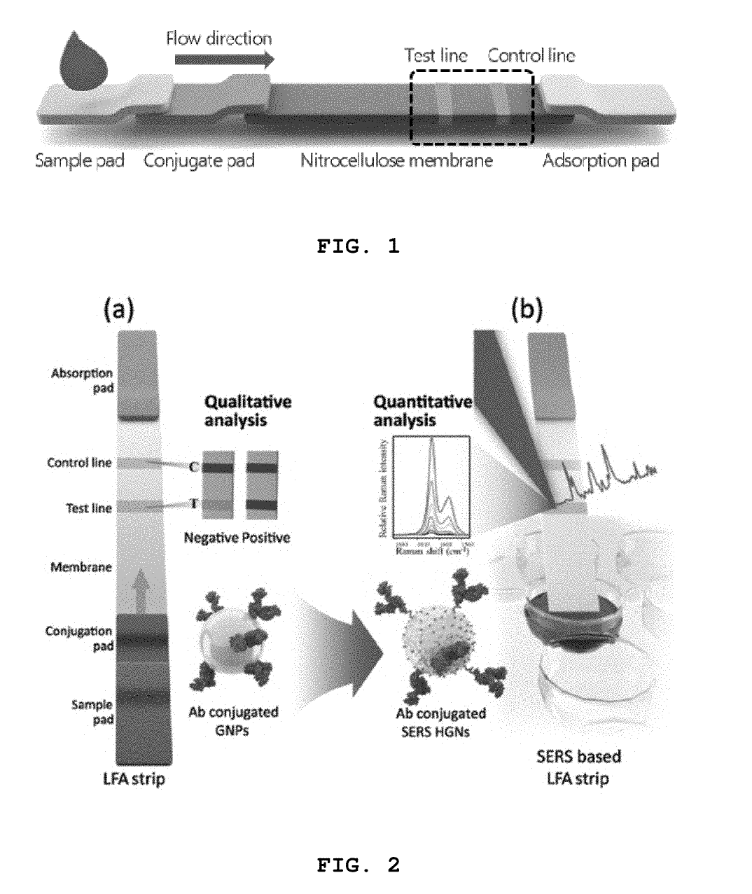

### DETAILED DESCRIPTION OF THE INVENTION

Hereinafter, specific examples are presented to help understanding the present disclosure. However, the following examples are given only as examples of the present disclosure and it will be obvious to those of ordinary skill in the art that various changes and modifications can be made within the scope of the present disclosure. Also, it will be obvious that such changes and modifications belong to the scope of the appended claims.

### <Example 1> Materials

HAuCl₄ (gold(III) chloride trihydrate), Na₃-citrate (trisodium citrate), DHLA (dihydrolipoic acid), EDC (1-ethyl,3-(3-dimethylaminopropyl)carbodiimide), NHS (4-(4-maleimidophenyl)butyric acid N-succinimidyl ester), CoCl₂ (ethanolamine, cobalt (II) chloride), BSA (bovine serum albumin), PVP (polyvinylpyrrolidone), tris-EDTA buffer (TE buffer, pH 8.0), S9008, rabbit anti-SEB (anti-staphylococcal enterotoxin B polyclonal antibody produced in rabbit) and anti-mouse IgG (anti-mouse IgG antibody produced in goat) were purchased from Sigma-Aldrich (St. Louis, Mo., USA). Surfactant G was purchased from Fitzgerald (Concord, Mass., USA). MGITC (malachite green isothiocyanate) was purchased from Invitrogen Corporation (Carlsbad, Calif., USA). S222 and Mouse anti-SEB (anti-staphylococcal enterotoxin B monoclonal antibody produced in mouse) were purchased from Santa Cruz Biotechnology (Santa Cruz, Calif., USA). SEB (recombinant enterotoxin type B for Staphylococcus aureus) was purchased from Cusabio (Wuhan, China). A NC (nitrocellulose) membrane-attached backing card (Hi-flow plus HF180) was purchased from Millipore Corporation (Billerica. Mass., USA). An absorption pad (CF3) was purchased from Whatman-GE Healthcare (Pittsburgh, Pa., USA).

### <Example 2> Preparation of SERS-Based Lateral Flow Immunoassay Kit

2-1: Synthesis of Hollow Gold Nanoparticle (HGN) and Immobilization (Conjugation) of Antibody

A hollow gold nanoparticle (HGN) was synthesized according to methods described in literatures (C. E. Talley, J. B. Jackson, C. Oubre, N. K. Grady, C. W. Hollars, S. M. Lane, T. R. Huser, P. Nordlander and N. J. Halas, Nano Lett., 2005, 5, 1569-1574; A. M. Schwartzberg, T. Y. Oshiro, J. Z. Zhang, T. Huser and C. E. Talley, Anal. Chem., 2006, 78, 4732-4736.; H. Chon, S. Lee, S.-Y. Yoon, E. K. Lee, S.-I. Chang and J. Choo, Chem. Commun., 2014, 50, 1058-1060.). Briefly, the hollow gold nanoparticle was synthesized by growing a gold nanoshell by reducing gold atoms on the surface of a cobalt nanoparticle used as a support and controlling the same. The cobalt nanoparticle was synthesized by reducing CoCl₂ with NaBH₄ under a N₂ purging condition. After adding a HAuCl₄ solution to the synthesized cobalt nanoparticle to induce nucleation of gold atoms in the solution, a thin shell enclosing the cobalt nanoparticle was grown. Then, the cobalt nanoparticle was completely dissolved to synthesize a hollow gold nanoparticle. The particle size and physical properties of the prepared hollow gold nanoparticle were evaluated by UV/Vis absorption spectroscopy, TEM (transmission electron microscopy) and DLS (dynamic light scattering) (FIG. 3).

The prepared hollow gold nanoparticle had a size of 45±12 nm and a thickness of 15±5 nm. A SERS nanoprobe was prepared from the hollow gold nanoparticle as follows. 5.0 μL of a MGITC Raman marker at a concentration of 10 μM was added to 1 mL of the hollow gold nanoparticle at a concentration of 0.1 nM and reaction was conducted for 30 minutes. The MGITC-adsorbed hollow gold nanoparticle was reacted for 30 minutes by adding 0.1 μL of 1.0 mM DHLA to substitute the surface of the nanoparticle with carboxyl groups. The carboxyl-substituted hollow gold nanoparticle was reacted for 1 hour by adding 1.0 μL of a 0.1 mM EDC/NHS solution. Then, reaction was conducted for 1 hour by adding 0.1 μL of 1.0 mg/mL mouse anti-SEB. After removing unreacted materials and antibodies through centrifugation, the unreacted portion on the surface of the hollow gold nanoparticle was inactivated by adding 0.5 μL of 1.0 mM ethanolamine. The prepared antibody-immobilized hollow gold nanoparticle was stored at 4° C. In order to improve the efficiency of reaction and diffusion of the nanoparticle in a lateral flow immunoassay (LFA) strip, a mixture of 20 μL of 10× concentration antibody- and MGITC-immobilized hollow gold nanoparticle, 20 μL of surfactant G (10%), 20 μL of PVP (10%) and 40 μL of TET buffer (Tween 20, 0.05 v/v %, pH 8.0) was used for a lateral flow immunoassay strip sensor.

2-2: Preparation of Lateral Flow Immunoassay (LFA) Strip

A lateral flow immunoassay strip consisted of a sample pad for sample injection, a conjugate pad on which the hollow gold nanoparticle is adsorbed, a nitrocellulose (NC) membrane as a detection pad and an absorption pad. In order to prepared the strip, a nitrocellulose membrane with a size of 3-10 μm was attached to a support (plastic backing card) and an absorption pad was attached to the end of the nitrocellulose membrane. A test line and a control line in the nitrocellulose membrane were prepared using 0.5 mg/mL rabbit anti-SEB and 0.1 mg/mL mouse anti-IgG. Each antibody was sprayed onto the nitrocellulose membrane at a concentration of 0.5 μL/cm using a precision line dispensing system (Zeta Corporation, South Korea). The antibody-sprayed nitrocellulose membrane was dried at room temperature for 1 hour. The nitrocellulose membrane having the antibodies adsorbed in the form of lines was cut to a thickness of 3.8 mm using a programmable cutter (Zeta Corporation, South Korea). Immunoassay using the prepared lateral flow immunoassay strip was conducted by dropping a sample onto a 96-well ELISA plate and then immersing the strip for simplification of the analytical procedure.

2-3: Methods for Detection and Analysis

The Raman spectra and SERS mapping images of the test line in the lateral flow immunoassay (LFA) strip were acquired using the inVia Raman microscope system (Renishaw, New Mills, United Kingdom). The inVia Raman microscope system uses a He—Ne laser with a power of 3 mW operating at a wavelength of 633 nm. The Rayleigh line was removed by placing a holographic notch filter in the collection path. Raman scattering was collected using a CCD (charge-coupled device) camera at a spectral resolution of 1 cm⁻¹. The Raman images were acquired by Raman point mapping using a 50× lens. The detection range was set using a stage that can be translated along x- and y-axes in micrometer scales and Raman signals were acquired from a total of 1600 pixels from a range of 200 μm (x-axis)×800 μm (y-axis) with a step size of 10 μm×10 μm. The SERS images obtained from the strip were corrected using the WiRE software V 4.0 (Renishaw, New Mills, United Kingdom) and the intensity of the Raman signal for each pixel was quantitatively analyzed using the peak of the Raman marker MGITC at 1615 cm⁻¹. The average spectrum of the SERS image of the lateral flow immunoassay strip at each concentration was obtained from the total pixels and quantitative analysis for different SEB concentrations was conducted based on this.

The physical properties of the prepared nanoparticle were analyzed using the Cary 100 spectrophotometer (Varian, Salt Lake City, Utah, USA) and the DLS (dynamic light scattering) Nano-ZS90 (Malvern). The shape and size of the prepared nanoparticle were identified from high-magnification TEM (transmission electron microscopy) images. The adsorption of the hollow nanoparticle in the lateral flow immunoassay strip was identified by SEM (scanning electron microscopy). Enzyme-linked immunosorbent assay (ELISA) was conducted for comparison with the immunoassay according to the present disclosure and calibration curves versus SEB concentration were constructed using a microplate reader (Power Wave X340, Bio-Tek, Winooski, Vt., USA). The Raman intensity of the test line in the lateral flow immunoassay strip at different SEB concentrations was identified using the Chemi-Doc imaging system (Bio-Rad, Hercules, Calif., USA).

### <Example 3> Detection of Target Material Using SERS-Based LFA Strip

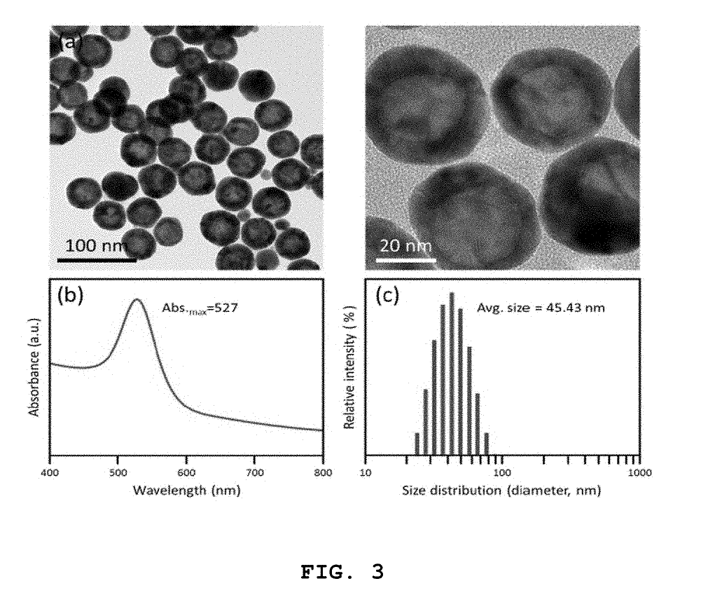

The operating principle of the surface-enhanced Raman scattering (SERS)-based high-sensitivity lateral flow immunoassay (LFA) strip is sandwich-type immunoassay. FIG. 2 (a) shows the operating principle of a general lateral flow immunoassay strip. An unknown sample containing a target material is dropped onto a sample pad of the LFA strip. The sample passes through a conjugate pad by a capillary phenomenon. An antibody-immobilized nanoparticle physically adsorbed to the conjugate pad undergoes an immune reaction with the target material in the sample. The resulting immune complex (antigen-gold nanoparticle) moves continuously toward a NC membrane by the capillary phenomenon and reaches a test line, where it undergoes a secondary immune reaction with an antibody adsorbed in the test line. A red line develops as the gold nanoparticle is accumulated in the test line. The excess antibody-conjugated gold nanoparticle moves further and is captured by an antibody adsorbed in a control region. Finally, two red lines appear if the target material is present (positive), and only one red line appears if the target material is absent (negative). The red line of the control region shows that the LFA strip works well.

### <Example 4> Quantitative Analysis Using SERS-Based LFA Strip

Although the existing LFA strip is commercialized and used as a POC (point-of-care) detection device, it has the problem that the sensitivity of analysis is low. This is a serious handicap in the early diagnosis of a disease. In addition, the existing lateral flow immunoassay sensor has the problem that quantitative analysis is impossible. The SERS-based LFA strip of the present disclosure overcomes these problems.

FIG. 2 (b) describes an analysis platform of the SERS-based LFA strip. The analytical method is identical to that of the existing lateral flow immunoassay sensor but there is a difference in the condition of the nanoparticle. The nanoparticle used in the SERS-based LFA strip is a hollow gold nanoparticle (HGN) on which a Raman marker is adsorbed. It is used for quantitative analysis of a target material (antigen) to be detected. In the presence of an antigen, the nanoparticle is accumulated in the test line and the test line develops a red line. Quantitative analysis of the concentration of the antigen is possible using the SERS signal from the Raman marker on the surface of the accumulated nanoparticle.

The method of immobilizing an antibody on the surface of the hollow gold nanoparticle may affect the flow in the strip because it can induce aggregation and instability of the nanoparticle. Therefore, the stability and flow ability of the nanoparticle in the strip were evaluated for immobilization of the antibody by physical adsorption and immobilization of the antibody by chemical reaction.

In Example 2-1, the antibody was immobilized by a chemical method. It is contrasted with physical adsorption. In the physical adsorption, the antibody is adsorbed on the surface of the hollow gold nanoparticle using electrostatic attraction. Details are as follows. After adding 1 μL of 1 mg/mL mouse anti-SEB to 1 mL of the prepared hollow gold nanoparticle, reaction was conducted for 1 hour. The surface of the hollow gold nanoparticle interacts with the antibody through electrostatic attraction. Unreacted residues were removed by centrifugation.

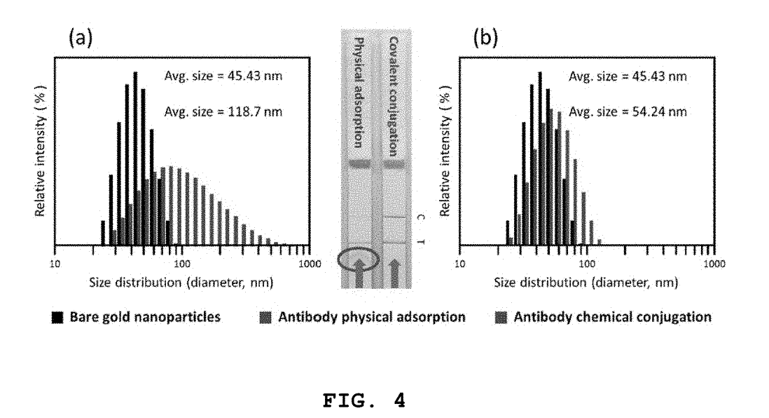

FIG. 4 shows DLS signals for the two types of antibody immobilization method (physical and chemical immobilization). The hollow gold nanoparticle on which the antibody is immobilized by physical adsorption (a) exhibits increased nonspecific aggregation of the nanoparticle and decreased efficiency of antibody reaction as compared to chemical immobilization. From comparison of FIGS. 4 (a) and (b), it was confirmed that the antibody immobilization by chemical substitution results in a narrower nanoparticle size distribution as well as a superior reaction efficiency without nonspecific aggregation or disturbed flow in the strip.

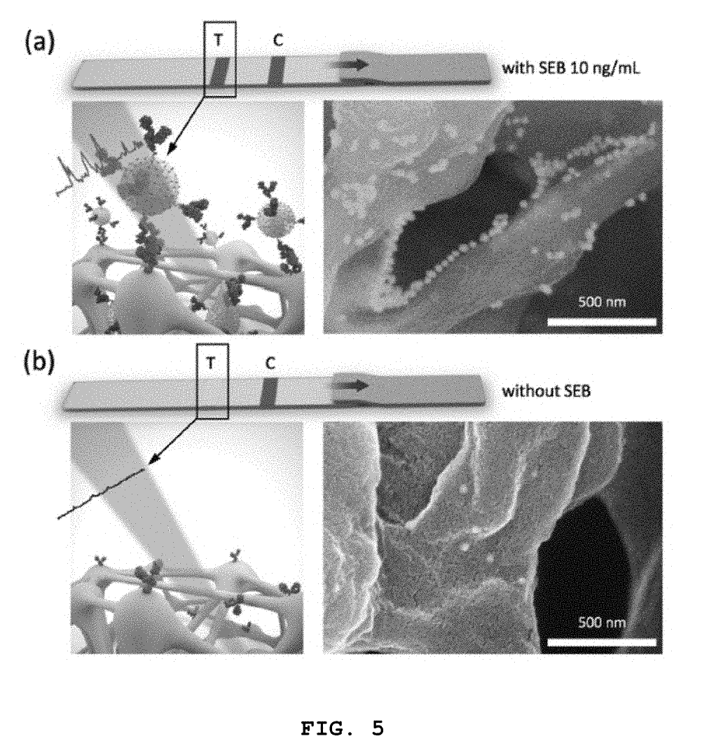

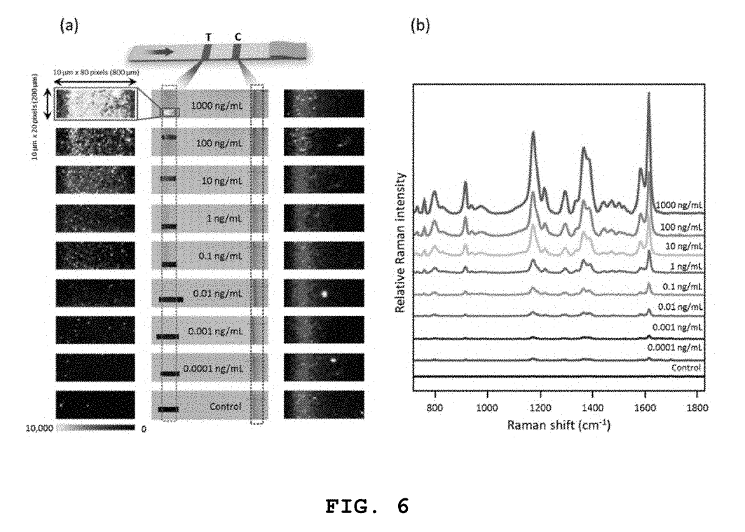

FIG. 5 shows the SEM image of the HGN immune complex formed in the test line depending on the presence or absence of the antigen (SEB). From FIG. 5 (a), it can be seen that the HGN immune complex forms clusters between the pores of the NC membrane when the antigen (SEB) is present (10 ng/mL). Due to these clusters, the test line turns red (“On”) and the SERS activity is exhibited with high sensitivity. In the absence of the antigen (SEB), as seen from FIG. 5 (b), no color change (“Off”) or SERS activity is observed because the immune complex is not formed in the test line. This is consistent with the color development of the test line depending on the antigen concentration. To conclude, when the SERS-based LFA strip of the present disclosure is used, the presence or absence of the target material can be detected visually as in the existing LFA strip. However, the SERS-based LFA strip of the present disclosure also enables quantitative analysis of the target material because the characteristic Raman signals of the SERS nanoprobe can be acquired depending on the concentration of the target material.

FIG. 6 (a) shows the SERS mapping result for the peak of the lateral flow immunoassay strip at 1615 cm⁻¹ for SEB at different concentrations (0-1000 ng/mL). Images were acquired from 80×20 pixels (1 pixel=10 μm×10 μm) at each concentration from 0 to 1000 ng/mL. The detection range of Raman point mapping image was set using a stage that can be translated along x- and y-axes in micrometer scales and Raman signals were acquired from a total of 1600 pixels from a range of 200 μm (x-axis)×800 μm (y-axis) with a step size of 10 μm×10 μm. The scale bar at the bottom left represents the SERS intensity, which is determined by the intensity of the peak at 1615 cm⁻¹. To conclude, it was confirmed that the amount of the immune complex formed in the test line increases as the SEB concentration changes from 0.1 pg/mL to 1000 ng/mL and this leads to increase in the SERS intensity.

However, the intensity was not uniform in the same region of the 1600 pixels of the mapping images due to the difference in nanoparticle aggregation and surface morphology depending on pixels. The average SERS intensity for the 1600 pixels of the strip was determined to solve this problem. FIG. 6 (b) shows the average spectrum of the 1600 pixels measured at different SEB concentrations. It can be seen that the intensity of the spectral signal (1615 cm⁻¹) increases with the concentration of the target material (antigen). The SERS mapping image of the control region was constant regardless of the SEB concentration.

### <Comparative Example 1> Detection of Target Material Using POC-Based LFA and ELISA

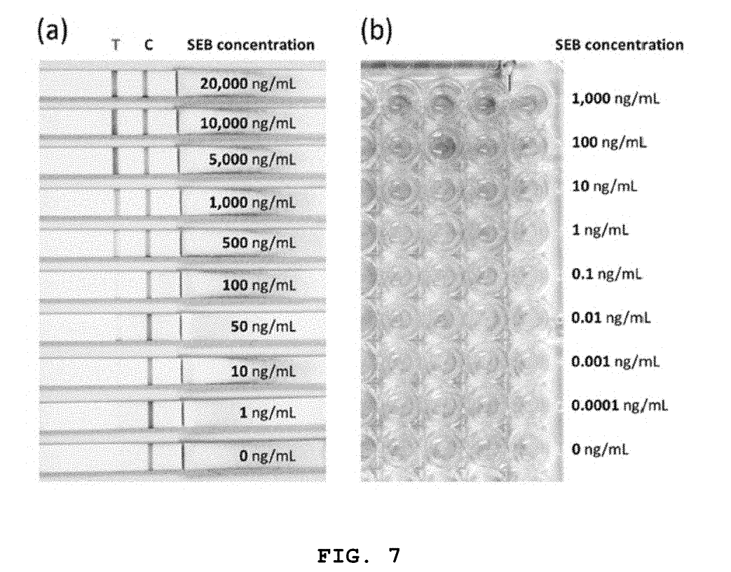

POC-based LFA and enzyme-linked immunosorbent assay (ELISA) were conducted to evaluate the detection sensitivity of the SERS-based LFA strip. The POC-based LFA refers to detecting the target material without conducting SERS measurement. 20 μL of a SEB solution was loaded on the LFA strip and passed through the absorption pad. Then, an antibody-conjugated HGN and a running buffer were loaded. The antibody-conjugated HGN formed a sandwich immune complex in the detection region by reacting with the SEB antigen. The remaining antibody-conjugated HGN reacted with a secondary antibody adsorbed at the control region. FIG. 7 (a) shows the change of the LFA strip at the SEB concentrations from 1 to 20,000 ng/mL. A red line was observed up to the SEB concentration of 10 ng/mL. Contrast images were measured using the Chemi-Doc imaging system in order to evaluate the optical density of the test line depending on the change in SEB concentration. The detection limit was found to be 10 ng/mL.

The enzyme-linked immunosorbent assay was conducted using the same antigen and antibody as those used for the SERS-based LFA strip. The capture antibody was immobilized on the surface of a 96-well plate and the remaining sites were treated with BSA to prevent nonspecific conjugation. Then, the SEB antigen was added for conjugation with the capture antibody. After washing 3 times with a micropipette, a detection antibody was added. For conjugation with the detection antibody, an enzyme-conjugated secondary antibody was added. Finally, a substrate was added to enable detection by an enzyme. FIG. 7 (b) shows the color change (from yellow to dark yellow) depending on the change in SEB concentration. The detection limit of the enzyme-linked immunosorbent assay using SEB was found to be 1.0 ng/mL.

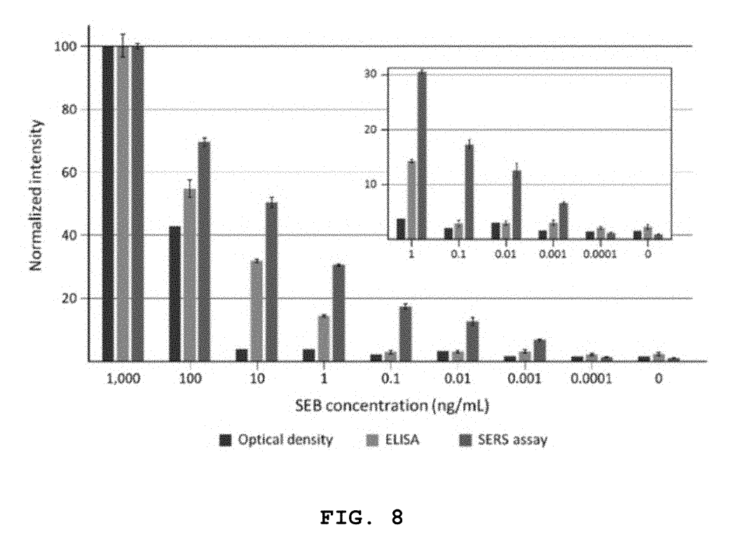

FIG. 8 compares a result of normalizing the result of the SERS-based LFA, POC-based LFA and ELISA for SEB concentrations changing from 10⁻⁴ to 10³ ng/mL. The SERS intensity was determined by the peak intensity of the Raman marker MGITC at 1615 cm⁻¹.

At large, the SERS-based LFA strip showed higher sensitivity of quantitative analysis as compared to other analytical methods. In particular, quantitative analysis was possible at concentrations of 1 ng/mL or lower unlike other analytical methods. This confirms higher sensitivity as compared to the existing POC-based LFA or ELISA. From the normalized curves depending on SEB concentration, the detection limits of the POC-based LFA strip (optical density), ELISA and SERS-based LFA strip were found to be 10, 1.0 and 0.001 ng/mL, respectively.

### <Example 4> Evaluation of Selectivity and Quantitative Analysis of SERS-Based LFA Strip

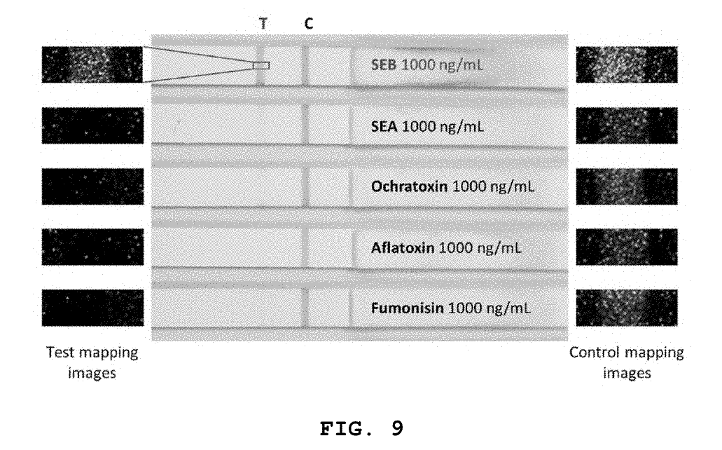

The selectivity of the SERS-based LFA strip according to the present disclosure was evaluated using five different toxoproteins at 1,000 ng/mL. FIG. 9 shows the immunoassay result for SEB, SEA (Staphylococcus aureus enterotoxin A) (Cusabio (Wuhan, China)), ochratoxin (Sigma-Aldrich (St. Louis, Mo., USA)), aflatoxin (Sigma-Aldrich (St. Louis, Mo., USA)) and fumonisin (Abcam (Cambridge, United Kingdom)) conducted using the SERS-based LFA strip. The detection region developed a red line in the presence of SEB only and the SERS mapping image was observed for SEB only. No positive response was detected for the toxoproteins other than SEB and the positive response was detected in the presence of SEB. That is to say, it was confirmed that the SERS-based LFA strip of the present disclosure shows high analytical selectivity.

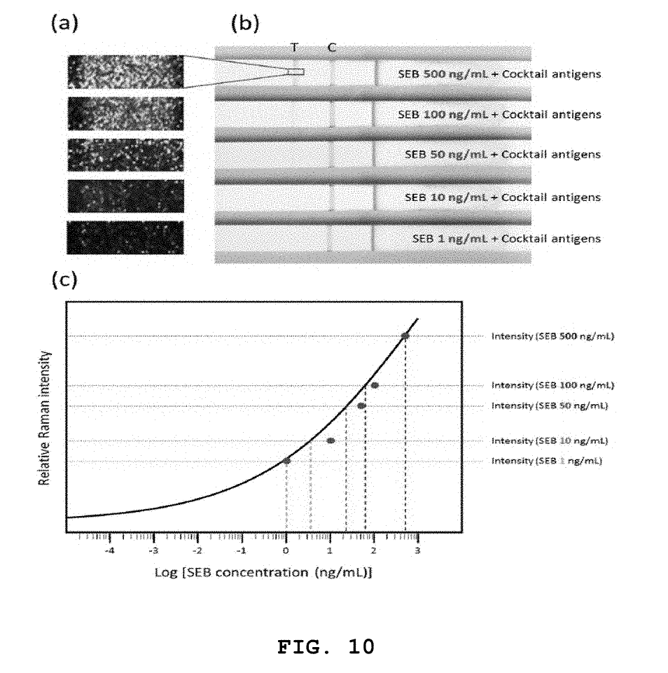

Selectivity and quantitative analysis tests were conducted with SEB at low concentrations (500, 100, 50, 10 and 1 ng/mL). The effect of nonspecific conjugation was tested using an antigen cocktail solution containing five different antigens (SEB, SEA, ochratoxin, aflatoxin and fumonisin at the same concentration of 100 ng/mL). The result is shown in FIG. 10. As seen from FIGS. 10 (a) and (b), the SERS-based LFA strip showed high selectivity only for SEB. The concentration of SEB in the antigen cocktail solution mixed with SEB at different concentrations was quantitated as shown in FIG. 10 (C).

### <Comparative Example 2> Comparative Analysis of SERS-Based LFA Strip Using Hollow Gold Nanoparticle (HGN) and SERS-Based LFA Strip Using Gold Nanoparticle (GNP)

The SERS-based LFA strip according to the present disclosure used HGN for SERS measurement. There are several other metal nanoparticles used as metal nanoprobes for SERS measurement in addition to the HGN. Among them, the gold nanoparticle (GNP) was selected for comparison of sensitivity with the SERS-based LFA strip HGN. Unlike HGN, GNP does not have a hollow cavity. The GNP was synthesized using a HAuCl₄ solution and trisodium citrate according to the method described in the literature (Frens, G. et al., 1973. Nat. Phys. Sci. 241, pp. 20-22). Briefly, a nanoparticle with a size of about 40 nm was synthesized by adding 500 μL of 1% trisodium citrate (Sigma-Aldrich) as a reducing agent to 50 mL of a boiling 0.01% HAuCl₄ solution (Sigma-Aldrich) and conduction reaction for 20 minutes.

A SERS nanoprobe and a SERS-based LFA strip using GNP were prepared under the same condition as the SERS-based LFA using HGN (see Example 2-2).

After preparing SERS-based LFA strips using HGN and GNP SERS nanoprobes, quantitative analysis was conducted using SEB as a target material at different SEB concentrations. Details are described in Table 1.

TABLE 1

| | | SERS-based |
|---|---|---|
| | SERS-based LFA using HGN | LFA using GNP |
| Detection | Hollow gold nanoparticle | Gold nanoparticle |
| probe (SERS | Raman marker: MGITC | Raman marker: MGITC |
| probe) | Anti-SEB antibody immobilized | Anti-SEB antibody |
| | | immobilized |
| Preparation | Prepared in the same manner as in Example 2-2 | |
| of LFA | Flow condition in LFA is identical | |
| Comparative | Quantitative analysis: comparison of Raman | |
| analysis | intensity of test line at different SEB concentrations | |
| Result | LOD: 0.001 ng/mL (1 pg/mL) | LOD: 0.1 ng/mL |
| | | (100 pg/mL) |

FIG. 11 compares the Raman intensity of the test line of the SERS-based LFA strip using HGN (a) and the SERS-based LFA strip using GNP (b) depending on the concentration of SEB. It can be seen that the two strips show decrease in Raman intensity s the SEB concentration is decreased. However, the LFA using HGN and the LFA using GNP show difference in Raman intensity at the same SEB concentration. At each concentration, the HGN-based LFA exhibits about 8-10 times higher Raman intensity than the GNP-based LFA. Accordingly, it was confirmed that the LFA using HGN has higher sensitivity than the GNP-based LFA.

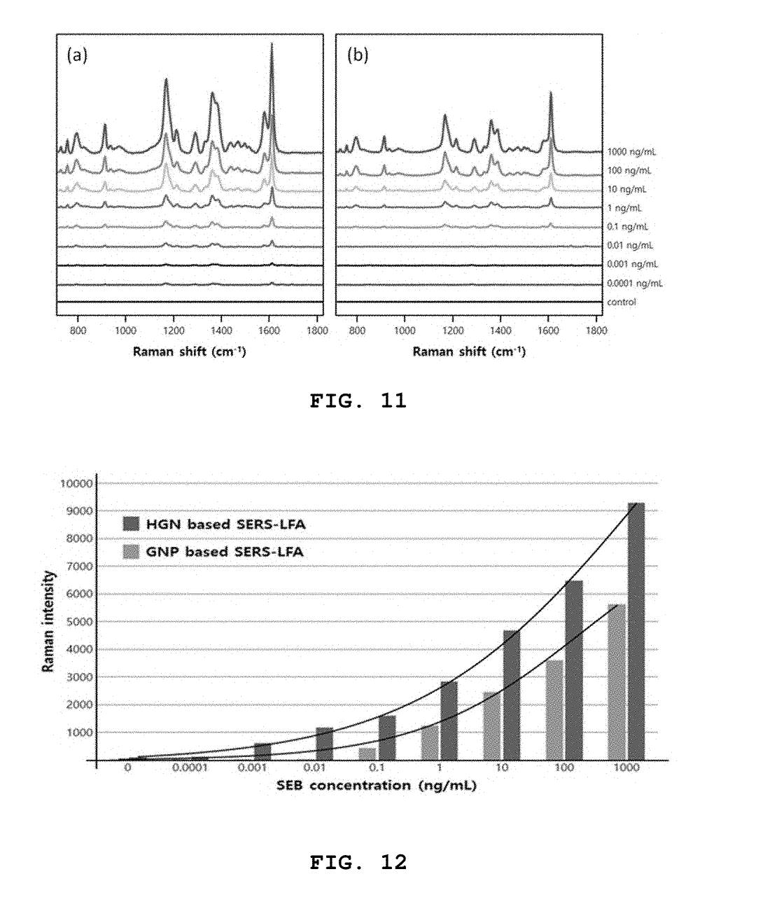

FIG. 12 shows the calibration curves for quantitative analysis of the SERS-based LFA strip using HGN (a) and the SERS-based LFA strip using GNP (b) depending on the concentration of SEB. The calibration curves of FIG. 12 were obtained by comparing the intensity of Raman signals depending on SEB concentration (FIG. 11). It was confirmed that the LFA using HGN shows change in Raman intensity as the SEB concentration changes from 1,000 to 0.001 ng/mL. The GNP-based LFA showed change in Raman intensity as the SEB concentration from 1,000 to 0.1 ng/mL. From the calibration curves, it was confirmed that the LFA using HGN has higher sensitivity than the GNP-based LFA.

In the present disclosure, the hollow metal nanoprobe with the Raman marker adsorbed was introduced to a lateral flow immunoassay sensor to overcome the low sensitivity of the existing lateral flow immunoassay sensor. As a result, high-sensitivity quantitative analysis was made possible through Raman mapping and imaging. For quantitative analysis and evaluation of sensitivity, the SEB toxoprotein was used as the target material and POC-based LFA and ELISA were selected as control groups for comparison. AS a result, it was confirmed that SEB could be detected with a high sensitivity of 0.001 ng/mL regardless of other toxoproteins. The result was 1,000-10,000 times superior as compared to the POC-based LFA or ELISA. In addition, it was confirmed that particularly the hollow metal nanoparticle from among the metal nanoprobes for SERS has high sensitivity. Accordingly, it is expected that the present disclosure is applicable to early diagnosis, environmental sensor, etc., to which the existing lateral flow immunoassay sensor is inapplicable.

1. A surface-enhanced Raman scattering (SERS)-based lateral flow immunoassay strip, comprising:
a sample pad into which a sample comprising a target material is introduced;
a conjugate pad comprising a hollow metal nanoprobe for surface-enhanced Raman scattering, on which an antibody that can be coupled to the target material and a Raman marker are immobilized;
and a detection pad comprising a detection region to which a secondary antibody that can be coupled to the target material coupled to the hollow metal nanoprobe is immobilized,
wherein the target material is detected by identifying color development at the detection pad and measuring a SERS signal.

2. The SERS-based lateral flow immunoassay strip according to claim 1, wherein the detection pad further comprises a control region to which an antibody that is coupled to the hollow metal nanoprobe is immobilized.

3. The SERS-based lateral flow immunoassay strip according to claim 1, wherein the hollow metal nanoprobe is a hollow gold nanoparticle.

4. The SERS-based lateral flow immunoassay strip according to claim 1, wherein the detection of the target material comprises qualitative analysis of identifying the presence of the target material through color development of the detection region and quantitative analysis of identifying the amount of the target material by measuring a SERS signal.

5. The SERS-based lateral flow immunoassay strip according to claim 1, wherein the detection limit of the target material is 0.001 ng/mL or lower.

6. A method for detecting a target material using the SERS-based lateral flow immunoassay strip according to claim 1, comprising:
a step of introducing a sample comprising a target material into a sample pad; and
a step of identifying color development at the detection pad and measuring a SERS signal.

7. The method for detecting a target material using the SERS-based lateral flow immunoassay strip according to claim 6, wherein the detection of the target material comprises qualitative analysis of identifying the presence of the target material through color development of a detection region of the detection pad and quantitative analysis of identifying the amount of the target material by measuring a SERS signal.

8. A SERS-based lateral flow immunoassay kit comprising:
the SERS-based lateral flow immunoassay strip according to claim 1; and
a SERS signal detector.
